(19) United States
(12) Patent Application Publication
(10) Pub. No.: US 2007/0148258 A1
(43) Pub. Date: Jun. 28, 2007

(54) Dialysates and methods and systems related thereto

(76) Inventors: W. Charles O'Neill, Decatur, GA (US); Koba Lomashvili, Atlanta, GA (US)

Correspondence Address: THOMAS, KAYDEN, HORSTEMEYER & RISLEY, LLP, 100 GALLERIA PARKWAY, NW, STE 1750, ATLANTA, GA, 30339-5948, US

(21) Appl. No.: 10/577,607

(22) PCT Filed: Oct. 27, 2004
(86) PCT No.: PCT/US04/35541

§ 371 Date: Apr. 27, 2006

Related U.S. Application Data
(60) Provisional application No. 60/515,174, filed on Oct. 28, 2003.

(51) Int. Cl. A61K 33/42

(52) U.S. Cl. 424/601; 604/19; 424/603

(57) ABSTRACT

Compositions, systems, and methods of treating conditions such as vascular calcification conditions are disclosed. A representative method includes administering to an individual in need of treatment an effective amount of at least one effector agent. Another method includes prophylactically treating vascular calcification or vascular calcification-related conditions by administering to an individual in need of treatment an effective amount of at least one effector agent. Still another method includes treating vascular calcification by administering an effective amount of at least one effector agent to an individual in need of treatment via hemodialysis.

### CROSS-REFERENCE TO RELATED APPLICATION

This application claims priority to copending U.S. provisional application entitled, “Pyrophosphate Dialysate Concentrate,” having Ser. No. 60/515,174, filed Oct. 28, 2003, which is entirely incorporated herein by reference.

### TECHNICAL FIELD OF THE INVENTION(S)

The present disclosure is generally related to compositions, systems, agents, and methods for administration to individuals and, more particularly, is related to compositions and agents designed for treatment of vascular calcification.

### BACKGROUND

Hemodialysis is a process by which microscopic soluble toxins are removed from the blood using a filtering membrane such as a dialyzer. Dialysis treatment replaces the function of the kidneys, which normally serve as the body's natural filtration system. Through the use of a blood filter and a chemical solution known as dialysate, the treatment removes waste products and excess fluids from the bloodstream, while maintaining the proper chemical balance of the blood. Its most prevalent application, however, is for patients with temporary or permanent kidney failure. For patients with end-stage renal disease (ESRD), whose kidneys are no longer capable of adequately removing fluids and wastes from their body or of maintaining the proper level of certain kidney-regulated chemicals in the bloodstream, dialysis is the only treatment option available outside of kidney transplantation.

Hemodialysis is the most frequently prescribed type of dialysis treatment in the United States. The treatment involves circulating the patient's blood outside of the body through an extracorporeal circuit (ECC), or dialysis circuit. Two needles are inserted into the patient's vein, or access site, and are attached to the ECC, which includes plastic blood tubing, a filter known as a dialyzer (artificial kidney), and a dialysis machine that monitors and maintains blood flow and administers dialysate. Small, unwanted compounds, e.g., toxins, diffuse from the blood into the dialysate solution, while larger compounds such as proteins are retained in the blood. Dialysate is a chemical bath that is used to draw waste products out of the blood. Since small molecules that are normal constituents of blood can also diffuse across the membrane, they are added to the dialysate to prevent their depletion. Typically, dialysate includes ions (e.g., Na⁺, K⁺, Cl⁻, Ca²⁺), buffer (HCO₃⁻), and glucose, preventing serious side effects that would result if blood levels of these important compounds were deleted in the hemodialysis process.

Since the 1980s, the majority of hemodialysis treatments in the United States have been performed with hollow fiber dialyzers. A hollow fiber dialyzer is composed of thousands of tube-like hollow fiber strands encased in a clear plastic cylinder several inches in diameter. There are two compartments within the dialyzer (the blood compartment and the dialysate compartment). The membrane that separates these two compartments is semipermeable. This means that it allows the passage of certain sized molecules across it, but prevents the passage of other, larger molecules. As blood is pushed through the blood compartment in one direction, suction or vacuum pressure pulls the dialysate through the dialysate compartment in a countercurrent, or opposite direction. These opposing pressures work to drain excess fluids out of the bloodstream and into the dialysate, a process called ultrafiltration.

A second process called diffusion moves waste products in the blood across the membrane into the dialysate compartment, where they are carried out of the body. At the same time, electrolytes and other chemicals in the dialysate solution cross the membrane into the blood compartment. The purified, chemically-balanced blood is then returned to the body.

Many of the risks and side effects associated with dialysis are a combined result of both the treatment and the poor physical condition of the ESRD patient. Current dialysis treatments have limited effectiveness and numerous serious unintended side effects. These treatments have progressed only incrementally since W. J. Kolff and H. Berk developed the first practical human hemodialysis machine in 1943.

One long-term side effect of hemodialysis and/or ESRD is deposition of calcium within blood vessels, known as vascular calcification. This calcification occurs in the media of large and small arteries in the matrix between smooth muscle cells, also known as Monckeberg's arteriosclerosis. Hyperphosphatemia is thought to underlie medial vascular calcification in advanced renal failure, but calcification can occur in other conditions in the absence of hyperphosphatemia, indicating that additional factors are also at play. A side effect of hyperphosphatemia is the formation of calcium-phosphate crystals in the blood and soft tissue.

Clinical practice to prevent medial vascular calcification in ESRD is based on the assumption that it is merely a manifestation of plasma concentrations of Ca²⁺ and PO₄³⁻ that are above the solubility product for Ca₃(PO₄)₂. However, abundant data indicate that this is not the entire explanation. Medial calcification is commonly seen in aging, and occurs in several genetic defects, all in the presence of normal plasma calcium and phosphate concentrations. These observations suggest that calcification can occur at normal plasma calcium and phosphate concentrations and that mechanisms to inhibit this are normally in place in individuals. Thus, vascular calcification can be considered as a failure of these inhibitory mechanisms.

In the prior art, there are no known methods for performing hemodialysis in a manner that reduces calcium deposition.

### SUMMARY

Briefly described, embodiments of the present disclosure include dialysates and methods and systems related to dialysates. Specifically, one exemplary method of the present disclosure includes providing vascular calcification therapy to an individual in need of treatment, wherein the provision of therapy includes administering to the individual an effective amount of pyrophosphate-type compound. Another exemplary method of the present disclosure includes hemodialyzing an individual in need thereof, wherein hemodialyzing includes diffusing dialysate comprising at least one pyrophosphate-type compound across a membrane in a hemodialysis system, and exposing the individual to an effective amount of the pyrophosphate-type compound.

The dialysates and compositions included in the present disclosure relate to pyrophosphate-type compounds. For example, the present disclosure includes a pharmaceutical composition that includes at least one pyrophosphate-type compound in combination with a pharmaceutically acceptable carrier, wherein the at least one pyrophosphate-type compound is present in a dosage level effective to treat vascular calcification. An additional exemplary compositions of the present disclosure are dialysate concentrates and dialysates that includes at least one pyrophosphate-type compound.

Also included in the present disclosure are systems for hemodialyzing patients. One exemplary system includes a blood compartment, a membrane in fluidic communication with the blood compartment, and a dialysate compartment, where the dialysate compartment includes a dialysate having a pyrophosphate-type compound.

Other systems, methods, features, and advantages of the present disclosure will be or will become apparent to one with skill in the art upon examination of the following drawings and detailed description. It is intended that all such additional systems, methods, features, and advantages be included within this description, be within the scope of the present disclosure, and be protected by the accompanying claims.

### BRIEF DESCRIPTION OF THE DRAWINGS

Many aspects of the disclosure can be better understood with reference to the following drawings. The components in the drawings are not necessarily to scale, emphasis instead being placed upon clearly illustrating the principles of the present disclosure.

FIG. 1 is an exemplary plot illustrating the plasma pyrophosphate concentrations in normal subjects (n=36) and in hemodialysis patients prior to dialysis (n=38). Bars indicate means.

FIG. 2 is an exemplary graph illustrating the results of in vitro dialysis of pyrophosphate. A 4 L solution of pyrophosphate in physiologic saline solution without calcium was circulated through a 2.1 m² cellulose acetate dialyzer at 400 ml/mm against a standard clinical bath without calcium. The concentration of pyrophosphate was measured at the times indicated. The line represents a single exponential fit.

FIG. 3 is an exemplary graph illustrating the change in plasma pyrophosphate concentration after hemodialysis. Samples were drawn immediately before and immediately after dialysis from the predialyzer tubing. The lines to the left and right indicate the mean values before and after dialysis respectively.

FIG. 4 is an exemplary graph illustrating the change in erythrocyte pyrophosphate content after hemodialysis. Plasma samples were drawn immediately before and immediately after dialysis from the predialyzer tubing and washed erythrocytes were extracted with HClO₄. The lines to the left and right indicate the mean values before and after dialysis respectively.

FIG. 5 is an exemplary bar chart illustrating the inhibition of vascular calcification by pyrophosphate. Specifically, FIG. 5 demonstrates the incorporation of calcium in aortas incubated for 9 days in DMEM containing 3.8 mM PO₄⁻³ with or without 12-20 units/ml of inorganic pyrophosphatase. Results shown are means of at least 10 aortic rings; where p<0.001 vs. control.

FIG. 6 is an exemplary micrograph of a slide that illustrates the histology of aortas incubated for 9 days with inorganic pyrophosphatase, shown with hematoxylin and eosin stain with luminal surface on the left and magnification at 400×.

FIG. 7 is an exemplary micrograph of a slide that illustrates the histology of aortas incubated for 9 days with inorganic pyrophosphatase, shown with von Kossa stain with luminal surface on the left and magnification at 400×.

FIG. 8 is an exemplary graph that illustrates the suppression of calcification in injured aortas by pyrophosphate. Injured aortas were incubated for 6 days in DMEM containing 3.8 mM PO₄⁻³ and varying concentrations of pyrophosphate. Results are means of at least 4 aortic rings.

FIG. 9 is a block diagram of an exemplary hemodialysis system that includes the disclosed compositions and can be used to perform the disclosed methods.

### DETAILED DESCRIPTION

The present disclosure may be understood more readily by reference to the following detailed description and the Examples included therein.

Before the present compounds, compositions, and methods are disclosed and described, it is to be understood that this disclosure is not limited to specific pharmaceutical carriers, or to particular pharmaceutical formulations or administration regimens, as such may, of course, vary. It is also to be understood that the terminology used herein is for the purpose of describing particular embodiments only and is not intended to be limiting.

Definitions

The term “individual” or “patient” refers to any living entity having at least one cell. A living organism can be as simple as, for example, a single eukaryotic cell or as complex as a mammal, including a human being.

The term “pyrophosphate” and “pyrophosphate-type compound” are used interchangeably throughout and refer to any compound or formulation including the chemical formula (P₂O₇)⁴⁻, which includes the acid anhydride formulation, as well as any salt or ester of pyrophosphoric acid. The term “ester” includes functional groups that have the general formula RCOOR, where the R's stand for the same or different aliphatic groups (e.g., alkyl groups, alkenyl groups, alkynl groups, cycloalkyl groups, cycloakenyl groups, etc.), aromatic groups (e.g., heterocyclic groups, aryl groups, etc.), and/or hydrogen ions. Examples of pyrophosphate salts are described in more detail in Kirk & Othmer, Encyclopedia of Chemical Technology, Second Edition, Volume 15, Interscience Publishers (1968). While these pyrophosphate salts serve as examples, the present disclosure is not limited only to the specific pyrophosphate salts listed by Kirk & Othmer.

The term “derivative” means a modification to the disclosed compounds including, but not limited to, hydrolysis, reduction, or oxidation products, of the disclosed compounds. Hydrolysis, reduction, and oxidation reactions are known in the art.

The term “therapeutically effective amount” as used herein refers to that amount of the compound being administered which will relieve to some extent one or more of the symptoms of the disorder being treated. In reference to vascular calcification or pathologies related to vascular calcification, a therapeutically effective amount refers to that amount which has the effect of: (1) reducing the amount of vascular calcification; (2) inhibiting (that is, slowing to some extent, and preferably stopping) vascular calcification; (3) preventing and/or reducing vascular calcification; (4) relieving to some extent (or, preferably, eliminating) one or more symptoms associated with a pathology related to or caused in part by vascular calcification; and/or (6) to prevent the chain of events downstream of an initial ischemic condition which leads to the pathology. By a “therapeutically effective amount” of one or more of the effector agents it is meant a sufficient amount of one or more of the effector agents to treat vascular calcification and vascular calcification-related conditions at a reasonable benefit/risk ratio applicable to any medical treatment. For example, a “therapeutically effective amount” of one or more of the effector agents is an amount sufficient to palliate, ameliorate, stabilize, reverse, slow, and/or delay the progression or onset of the disease state compared to not administering one or more of the effector agents.

It will be understood, however, that the total daily usage of the effector agents of the present disclosure will be decided by the attending physician within the scope of sound medical judgment. The specific therapeutically effective dose level for any particular individual will depend upon a variety of factors, including for example, the disorder being treated and the severity of the disorder; activity of the specific effector agents employed; the specific effector agents employed, the age, body weight, general health, sex and diet of the patient; the time of administration; route of administration; rate of excretion of the specific effector agents employed; the duration of the treatment; drugs used in combination or coincidental with the specific composition employed; and like factors well known in the medical arts. For example, it is well within the skill of the art to start doses of the effector agents at levels lower than those required to achieve the desired therapeutic effect and to gradually increase the dosage until the desired effect is achieved.

Effector agents are preferably formulated in dosage unit form for ease of administration and uniformity of dosage. “Dosage unit form” as used herein refers to a physically discrete unit of the effector agents appropriate for the individual to be treated. Each dosage should contain the quantity of effector agents calculated to produce the desired therapeutic effect either as such, or in association with the selected pharmaceutical carrier medium. Preferred unit dosage formulations are those containing a dose or unit, daily sub-dose, or an appropriate fraction thereof normally administered in one dialysis treatment session, of the administered effector agent. In this regard, studies were performed to assess the dosage regimen for pyrophosphate (PPi) compounds.

Effector agents and compositions (hereinafter “effector agents”) of this disclosure can be used to treat conditions such as, but not limited to, vascular calcification and vascular calcification-related diseases. In addition, effector agents of this disclosure can be used prophylactically to inhibit the development and/or slow the development of the vascular calcification and vascular calcification-related conditions and/or advanced stages of vascular calcification and vascular calcification-related conditions. Effector agents of the present disclosure may be used as the active ingredient in combination with one or more pharmaceutically acceptable carrier mediums and/or excipients.

“Pharmaceutically acceptable salt” refers to those salts which retain the biological effectiveness and properties of the free bases and which are obtained by reaction with inorganic or organic acids such as, but not limited to, hydrochloric acid, hydrobromic acid, sulfuric acid, nitric acid, phosphoric acid, methanesulfonic acid, ethanesulfonic acid, p-toluenesulfonic acid, salicylic acid, malic acid, maleic acid, succinic acid, tartaric acid, citric acid, and the like. By “pharmaceutically acceptable salt” it is meant those salts which are, within the scope of sound medical judgement, suitable for use in contact with the tissues of individuals without undue toxicity, irritation, allergic response and the like, and are commensurate with a reasonable benefit/risk ratio and effective for their intended use. The salts can be prepared in situ during the final isolation and purification of one or more effector agents, or separately by reacting the free base function with a suitable organic acid.

The term “pharmaceutically acceptable esters” as used herein refers to those esters of one or more effector agents which are suitable, within the scope of sound medical judgement, for use in contact with the tissues of individuals without undue toxicity, irritation, allergic response, and the like, are commensurate with a reasonable benefit/risk ratio, and are effective for their intended use.

The term “pharmaceutically acceptable prodrugs” as used herein refers to those prodrugs of one or more effector agents which are, within the scope of sound medical judgement, suitable for use in contact with the tissues of individuals without undue toxicity, irritation, allergic response, and the like, are commensurate with a reasonable benefit/risk ratio, and are effective for their intended use. Pharmaceutically acceptable prodrugs also include zwitterionic forms, where possible, of one or more compounds of the composition. The term “prodrug” refers to compounds that are rapidly transformed in vivo to yield the parent compound, for example by hydrolysis in blood.

A “pharmaceutical composition” refers to a mixture of one or more of the compounds described herein, or pharmaceutically acceptable salts thereof, with other chemical components, such as physiologically acceptable carriers and excipients. One purpose of a pharmaceutical composition is to facilitate administration of a compound to an organism.

As used herein, a “pharmaceutically acceptable carrier” refers to a carrier or diluent that does not cause significant irritation to an organism and does not abrogate the biological activity and properties of the administered compound.

As used herein, “pharmaceutically acceptable carrier medium” includes any and all carriers, solvents, diluents, or other liquid vehicles, dispersion or suspension aids, surface active agents, isotonic agents, thickening or emulsifying agents, preservatives, solid binders, lubricants, adjuvants, vehicles, delivery systems, disintegrants, absorbents, preservatives, surfactants, colorants, flavorants, or sweeteners and the like, as suited to the particular dosage form desired. Preferably, the pharmaceutically acceptable carrier medium is dialysate.

An “excipient” refers to an inert substance added to a pharmaceutical composition to further facilitate administration of a compound. Examples of excipients include, but are not limited to, calcium carbonate, calcium phosphate, various sugars and types of starch, cellulose derivatives, gelatin, vegetable oils, and polyethylene glycols.

“Treating” or “treatment” of a disease includes preventing the disease from occurring in an animal that may be predisposed to the disease but does not yet experience or exhibit symptoms of the disease (prophylactic treatment), inhibiting the disease (slowing or arresting its development), providing relief from the symptoms or side-effects of the disease (including palliative treatment), and relieving the disease (causing regression of the disease). With regard to vascular calcification, these terms simply mean that the life expectancy of an individual affected with vascular calcification will be increased or that one or more of the symptoms of the disease will be reduced.

The term “prodrug” refers to an agent that is converted into a biologically active form in vivo. Prodrugs are often useful because, in some situations, they may be easier to administer than the parent compound. They may, for instance, be bioavailable by oral administration whereas the parent compound is not. The prodrug may also have improved solubility in pharmaceutical compositions over the parent drug. A prodrug may be converted into the parent drug by various mechanisms, including enzymatic processes and metabolic hydrolysis. Harper, N. J. (1962). Drug Latentiation in Jucker, ed. Progress in Drug Research, 4:221-294; Morozowich et al. (1977). Application of Physical Organic Principles to Prodrug Design in E. B. Roche ed. Design of Biopharmaceutical Properties through Prodrugs and Analogs, APhA; Acad. Pharm. Sci.; E. B. Roche, ed. (1977). Bioreversible Carriers in Drug in Drug Design, Theory and Application, APhA; H. Bundgaard, ed. (1985) Design of Prodrugs, Elsevier; Wang et al. (1999) Prodrug approaches to the improved delivery of peptide drug, Curr. Pharm. Design 5(4):265-287; Pauletti et al. (1997). Improvement in peptide bioavailability: Peptidomimetics and Prodrug Strategies, Adv. Drug. Delivery Rev. 27:235-256; Mizen et al. (1998). The Use of Esters as Prodrugs for Oral Delivery of β-Lactam antibiotics, Pharm. Biotech. 11:345-365; Gaignault et al. (1996). Designing Prodrugs and Bioprecursors I. Carrier Prodrugs, Pract. Med. Chem. 671-696; M. Asgharnejad (2000). Improving Oral Drug Transport Via Prodrugs, in G. L. Amidon, P. I. Lee and E. M. Topp, Eds., Transport Processes in Pharmaceutical Systems, Marcell Dekker, p. 185-218; Balant et al. (1990) Prodrugs for the improvement of drug absorption via different routes of administration, Eur. J. Drug Metab. Pharmacokinet., 15(2): 143-53; Balimane and Sinko (1999). Involvement of multiple transporters in the oral absorption of nucleoside analogues, Adv. Drug Delivery Rev., 39(1-3):183-209; Browne (1997). Fosphenyloin (Cerebyx), Clin. Neuropharmacol. 20(1): 1-12; Bundgaard (1979). Bioreversible derivatization of drugs—principle and applicability to improve the therapeutic effects of drugs, Arch. Pharm. Chemi. 86(1): 1-39; H. Bundgaard, ed. (1985) Design of Prodrugs, New York: Elsevier; Fleisher et al. (1996). Improved oral drug delivery: solubility limitations overcome by the use of prodrugs, Adv. Drug Delivery Rev. 19(2): 115-130; Fleisher et al. (1985). Design of prodrugs for improved gastrointestinal absorption by intestinal enzyme targeting, Methods Enzymol. 112: 360-81; Farquhar D, et al. (1983). Biologically Reversible Phosphate-Protective Groups, J. Pharm. Sci., 72(3): 324-325; Han, H. K. et al. (2000). Targeted prodrug design to optimize drug delivery, AAPS Pharm Sci., 2(1): E6; Sadzuka Y. (2000). Effective prodrug liposome and conversion to active metabolite, Curr. Drug Metab., 1(1):31-48; D. M. Lambert (2000). Rationale and applications of lipids as prodrug carriers, Eur. J. Pharm. Sci., 11 Suppl 2:S15-27; Wang, W. et al. (1999). Prodrug approaches to the improved delivery of peptide drugs. Curr. Pharm. Des., 5(4):265-87.

The terms “alk” or “alkyl” refer to straight or branched chain hydrocarbon groups having 1 to 12 carbon atoms, preferably 1 to 8 carbon atoms, such as methyl, ethyl, n-propyl, i-propyl, n-butyl, i-butyl, t-butyl, pentyl, hexyl, heptyl, octyl, etc. Lower alkyl groups, that is, alkyl groups of 1 to 6 carbon atoms, are generally most preferred. The term “substituted alkyl” refers to alkyl groups substituted with one or more groups, preferably selected from aryl, substituted aryl, heterocyclo, substituted heterocyclo, carbocyclo, substituted carbocyclo, halo, hydroxy, alkoxy (optionally substituted), aryloxy (optionally substituted), alkylester (optionally substituted), arylester (optionally substituted), alkanoyl (optionally substituted), aryol (optionally substituted), cyano, nitro, amino, substituted amino, amido, lactam, urea, urethane, sulfonyl, etc.

The term “alkoxy” means an alkyl group linked to oxygen thus: R—O—. In this function, R represents the alkyl group. An example would be the methoxy group CH₃O—.

The term “alkenyl” refers to straight or branched chain hydrocarbon groups having 2 to 12 carbon atoms, preferably 2 to 4 carbon atoms, and at least one double carbon to carbon bond (either cis or trans), such as ethenyl. The term “substituted alkenyl” refers to alkenyl groups substituted with one or more groups, preferably selected from aryl, substituted aryl, heterocyclo, substituted heterocyclo, carbocyclo, substituted carbocyclo, halo, hydroxy, alkoxy (optionally substituted), aryloxy (optionally substituted), alkylester (optionally substituted), arylester (optionally substituted), alkanoyl (optionally substituted), aryol (optionally substituted), cyano, nitro, amino, substituted amino, amido, lactam, urea, urethane, sulfonyl, etc.

The term “alkynyl” refers to straight or branched chain hydrocarbon groups having 2 to 12 carbon atoms, preferably 2 to 4 carbon atoms, and at least one triple carbon to carbon bond, such as ethynyl. The term. “substituted alkynyl” refers to alkynyl groups substituted with one or more groups, preferably selected from aryl, substituted aryl, heterocyclo, substituted heterocyclo, carbocyclo, substituted carbocyclo, halo, hydroxy, alkoxy (optionally substituted), aryloxy (optionally substituted), alkylester (optionally substituted), arylester (optionally substituted), alkanoyl (optionally substituted), aryol (optionally substituted), cyano, nitro, amino, substituted amino, amido, lactam, urea, urethane, sulfonyl, etc.

The terms “ar” or “aryl” refer to aromatic homocyclic (e.g., hydrocarbon) mono-, bi- or tricyclic ring-containing groups preferably having 6 to 12 members such as phenyl, naphthyl and biphenyl. Phenyl is a preferred aryl group. The term “substituted aryl” refers to aryl groups substituted with one or more groups, preferably selected from alkyl, substituted alkyl, alkenyl (optionally substituted), aryl (optionally substituted), heterocyclo (optionally substituted), halo, hydroxy, alkoxy (optionally substituted), aryloxy (optionally substituted), alkanoyl (optionally substituted), aroyl, (optionally substituted), alkylester (optionally substituted), arylester (optionally substituted), cyano, nitro, amino, substituted amino, amido, lactam, urea, urethane, sulfonyl, etc., where optionally one or more pair of substituents together with the atoms to which they are bonded form a 3 to 7 member ring.

The terms “cycloalkyl” and “cycloalkenyl” refer to mono-, bi- or tri homocylcic ring groups of 3 to 15 carbon atoms which are, respectively, fully saturated and partially unsaturated. The term “cycloalkenyl” includes bi- and tricyclic ring systems that are not aromatic as a whole, but contain aromatic portions (e.g., fluorene, tetrahydronapthalene, dihydroindene, and the like). The rings of multi-ring cycloalkyl groups may be either fused, bridged and/or joined through one or more spiro unions. The terms “substituted cycloalkyl” and “substituted cycloalkenyl” refer, respectively, to cycloalkyl and cycloalkenyl groups substituted with one or more groups, preferably selected from aryl, substituted aryl, heterocyclo, substituted heterocyclo, carbocyclo, substituted carbocyclo, halo, hydroxy, alkoxy (optionally substituted), aryloxy (optionally substituted), alkylester (optionally substituted), arylester (optionally substituted), alkanoyl (optionally substituted), aryol (optionally substituted), cyano, nitro, amino, substituted amino, amido, lactam, urea, urethane, sulfonyl, etc.

The terms “carbocyclo”, “carbocyclic” or “carbocyclic group” refer to both cycloalkyl and cycloalkenyl groups. The terms “substituted carbocyclo”, “substituted carbocyclic” or “substituted carbocyclic group” refer to carbocyclo or carbocyclic groups substituted with one or more groups as described in the definition of cycloalkyl and cycloalkenyl.

The terms “halogen” and “halo” refer to fluorine, chlorine, bromine, and iodine.

The terms “heterocycle”, “heterocyclic”, “heterocyclic group” or “heterocyclo” refer to fully saturated or partially or completely unsaturated, including aromatic (“heteroaryl”) or nonaromatic cyclic groups (for example, 3 to 13 member monocyclic, 7 to 17 member bicyclic, or 10 to 20 member tricyclic ring systems, preferably containing a total of 3 to 10 ring atoms) which have at least one heteroatom in at least one carbon atom-containing ring. Each ring of the heterocyclic group containing a heteroatom may have 1, 2, 3 or 4 heteroatoms selected from nitrogen atoms, oxygen atoms and/or sulfur atoms, where the nitrogen and sulfur heteroatoms may optionally be oxidized and the nitrogen heteroatoms may optionally be quaternized. The heterocyclic group may be attached at any heteroatom or carbon atom of the ring or ring system. The rings of multi-ring heterocycles may be fused, bridged and/or joined through one or more spiro unions.

Exemplary monocyclic heterocyclic groups include azetidinyl, pyrrolidinyl, pyrrolyl, pyrazolyl, oxetanyl, pyrazolinyl, imidazolyl, imidazolinyl, imidazolidinyl, oxazolyl, oxazolidinyl, isoxazolinyl, isoxazolyl, thiazolyl, thiadiazolyl, thiazolidinyl, isothiazolyl, isothiazolidinyl, furyl, tetrahydrofuryl, thienyl, oxadiazolyl, piperidinyl, piperazinyl, 2-oxopiperazinyl, 2-oxopiperidinyl, 2-oxopyrrolodinyl, 2-oxoazepinyl, azepinyl, 4-piperidonyl, pyridyl, pyrazinyl, pyrimidinyl, pyridazinyl, triazinyl, tetrahydropyranyl, tetrazoyl, triazolyl, morpholinyl, thiamorpholinyl, thiamorpholinyl sulfoxide, thiamorpholinyl sulfone, 1,3-dioxolane and tetrahydro-1,1-dioxothienyl, and the like.

Exemplary bicyclic heterocyclic groups include indolyl, benzothiazolyl, benzoxazolyl, benzothienyl, quinuclidinyl, quinolinyl, tetra-hydroisoquinolinyl, isoquinolinyl, benzimidazolyl, benzopyranyl, indolizinyl, benzofuryl, benzofuranly, dihydrobenzofuranyl, chromonyl, coumarinyl, benzodioxolyl, dihydrobenzodioxolyl, benzodioxinyl, cinnolinyl, quinoxalinyl, indazolyl, pyrrolopyridyl, furopyridinyl (such as furo[2,3-c]pyridinyl, furo[3,2-b]pyridinyl), or furo[2,3-b]pyridinyl), dihydroisoindolyl, dihydroquinazolinyl (such as 3,4-dihydro-4-oxo-quinazolinyl), tetrahydroquinolinyl, azabicycloalkyls (such as 6-azabicyclo[3.2.1]octane), azaspiroalkyls (such as 1,4 dioxa-8-azaspiro[4.5]decane), imidazopyridinyl (such as imidazo[1,5-a]pyridin-3-yl), triazolopyridinyl (such as 1,2,4-triazolo[4,3-a]pyridin-3-yl), and hexahydroimidazopyridinyl (such as 1,5,6,7,8,8a-hexahydroimidazo[1,5-a]pyridin-3-yl), and the like.

Exemplary tricyclic heterocyclic groups include carbazolyl, benzidolyl, phenanthrolinyl, acridinyl, phenanthridinyl, xanthenyl and the like.

The terms “substituted heterocycle”, “substituted heterocyclic”, “substituted heterocyclic group” and “substituted heterocyclo” refer to heterocycle, heterocyclic and heterocyclo groups substituted with one or more groups preferably selected from alkyl, substituted alkyl, alkenyl, oxo, aryl, substituted aryl, heterocyclo, substitute heterocyclo, carbocyclo (optionally substituted), halo, hydroxy, alkoxy (optionally substituted), aryloxy (optionally substituted), alkanoyl (optionally substituted), aroyl (optionally substituted), alkylester (optionally substituted), arylester (optionally substituted), cyano, nitro, amido, amino, substituted amino, lactam, urea, urethane, sulfonyl, etc., where optionally one or more pair of substituents together with the atoms to which they are bonded form a 3 to 7 member ring.

The term “alkanoyl” refers to alkyl group (which may be optionally substituted as described above) linked to a carbonyl group (i.e., —C(O)-alkyl). Similarly, the term “aroyl” refers to an aryl group (which may be optionally substituted as described above) linked to a carbonyl group (i.e., —C(O)-aryl).

Throughout the specification, groups and substituents thereof may be chosen to provide stable moieties and compounds.

The disclosed compounds form salts that are also within the scope of this disclosure. Reference to a compound of any of the formulas herein is understood to include reference to salts thereof, unless otherwise indicated. The term “salt(s)”, as employed herein, denotes acidic and/or basic salts formed with inorganic and/or organic acids and bases. In addition, when a compound of either Formula I or II (given below) contains both a basic moiety and an acidic moiety, zwitterions (“inner salts”) may be formed and are included within the term “salt(s)” as used herein. Pharmaceutically acceptable (e.g., non-toxic, physiologically acceptable) salts are preferred, although other salts are also useful (e.g., in isolation or purification steps which may be employed during preparation). Salts of the compounds of either Formula I or II can be formed, for example, by reacting a compound with an amount of acid or base, such as an equivalent amount, in a medium such as one in which the salt precipitates or in an aqueous medium followed by lyophilization.

The disclosed compounds that contain a basic moiety may form salts with a variety of organic and inorganic acids. Exemplary acid addition salts include acetates (such as those formed with acetic acid or trihaloacetic acid, for example, trifluoroacetic acid), adipates, alginates, ascorbates, aspartates, benzoates, benzenesulfonates, bisulfates, borates, butyrates, citrates, camphorates, camphorsulfonates, cyclopentanepropionates, digluconates, dodecylsulfates, ethanesulfonates, fumarates, glucoheptanoates, glycerophosphates, hemisulfates, heptanoates, hexanoates, hydrochlorides (formed with hydrochloric acid), hydrobromides (formed with hydrogen bromide), hydroiodides, 2-hydroxyethanesulfonates, lactates, maleates (formed with maleic acid), methanesulfonates (formed with methanesulfonic acid), 2-naphthalenesulfonates, nicotinates, nitrates, oxalates, pectinates, persulfates, 3-phenylpropionates, phosphates, picrates, pivalates, propionates, salicylates, succinates, sulfates (such as those formed with sulfuric acid), sulfonates (such as those mentioned herein), tartrates, thiocyanates, toluenesulfonates such as tosylates, undecanoates, and the like.

The disclosed compounds that contain an acidic moiety may form salts with a variety of organic and inorganic bases. Exemplary basic salts include ammonium salts, alkali metal salts such as sodium, lithium, and potassium salts, alkaline earth metal salts such as calcium and magnesium salts, salts with organic bases (e.g., organic amines) such as benzathines, dicyclohexylamines, hydrabamines (formed with N,N-bis(dehydroabietyl)ethylenediamine), N-methyl-D-glucamines, N-methyl-D-glucamides, t-butyl amines, and salts with amino acids such as arginine, lysine, and the like.

Basic nitrogen-containing groups may be quaternized with agents such as lower alkyl halides (e.g., methyl, ethyl, propyl, and butyl chlorides, bromides and iodides), dialkyl sulfates (e.g., dimethyl, diethyl, dibutyl, and diamyl sulfates), long chain halides (e.g., decyl, lauryl, myristyl and stearyl chlorides, bromides and iodides), aralkyl halides (e.g., benzyl and phenethyl bromides), and others.

Solvates of the compounds of the disclosure are also contemplated herein. Solvates of the compounds are preferably hydrates.

To the extent that the disclosed compounds, and salts thereof, may exist in their tautomeric form, all such tautomeric forms are contemplated herein as part of the present disclosure.

All stereoisomers of the present compounds, such as those which may exist due to asymmetric carbons on the various substituents, including enantiomeric forms (which may exist even in the absence of asymmetric carbons) and diastereomeric forms, are contemplated within the scope of this disclosure. Individual stereoisomers of the compounds of the disclosure may, for example, be substantially free of other isomers, or may be admixed, for example, as racemates or with all other, or other selected, stereoisomers. The chiral centers of the compounds of the present disclosure can have the S or R configuration as defined by the IUPAC 1974 Recommendations.

The terms “including”, “such as”, “for example”, and the like, are intended to refer to exemplary embodiments and not to limit the scope of the present disclosure.

The present disclosure provides compositions and agents that can be used to treat individuals having vascular calcification and vascular calcification-related conditions. In addition, the present disclosure provides compositions and methods of treating individuals that are predisposed to vascular calcification and vascular calcification-related conditions. The compositions include at least one pyrophosphate-type compound.

Pyrophosphate-type compounds can include, but are not limited to, the structure of Formula I illustrated below: 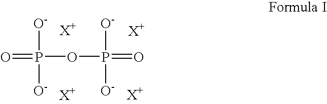
More particularly, pyrophosphate-type compounds can include any number of cations X+ or substituents ionically bonded to or in free association with the oxygen anions (O⁻). Examples of cations X include, but are not limited to, Li, Na, K, Ca, Mg, Cr, Mn, Fe, and/or Zn. Each of the X cations can be the same or different from the other X cations. For example, the pyrophosphate-type compound can be tetraalkali metal pyrophosphate, dialkali metal diacid pyrophosphate, trialkali metal monoacid pyrophosphate, or mixtures thereof. Specifically, the pyrophosphate-type compound can be, for example, tetrasodium pyrophosphate, tetrapotassium pyrophosphate, dicalcium pyrophosphate, phosphoric acid, sodium acid pyrophosphate, sodium dihydrogen pyrophosphate, or mixtures thereof.

The pyrophosphate-type compounds can also include the following structure of Formula II: 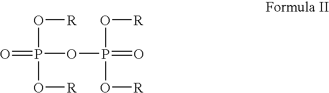
where exemplary functional groups of the pyrophosphate-type compounds are indicated as R. Each of the functional groups R can individually include, but are not limited to, hydrogen, alkyl groups, aryl groups, halo groups (F, Cl, Br, and I) hydroxy groups, alkoxy groups, alkylamino groups, dialkylamino groups, acyl groups, carboxyl groups, carboamido groups, sulfonamide groups, aminoacyl groups, amide groups, amine groups, nitro groups, organo selenium compounds, hydrocarbons, cyclic hydrocarbons, hydrogen, nitrogen, oxygen, sulphur, NR, and CR. Each of the R functional groups can be the same or different from the other R functional groups.

Where such forms exist, pyrophosphate-type compounds can include, but are not limited to, pyrophosphate derivatives that function to treat vascular calcification and vascular calcification-related conditions in an individual, and/or function prophylactically. In addition, where such forms exist, pyrophosphate-type compounds can include pharmaceutically acceptable salts, esters, and prodrugs of the pyrophosphate-type compounds described or referred to above.

Included in the present disclosure are dialysates that include at least one pyrophosphate-type compound as described above. One exemplary dialysate includes a pyrophosphate concentration of at least about 1 μM. Specifically, the pyrophosphate concentration can be about 1 μM to about 10 μM, or from about 3 μM to about 5 μM.

Included in the present disclosure are dialysate concentrates that include at least one pyrophosphate-type compound as described above. One exemplary dialysate concentrate includes a pyrophosphate concentration of about 50 μM to about 1 mM.

Included in the present disclosure are methods of providing vascular calcification therapy to an individual in need of treatment. One such exemplary method includes administering to the individual a therapeutically effective amount of a pyrophosphate-type compound. When used in the above or other treatments, a therapeutically effective amount of one or more of the effector agents may be employed in pure form or, where such forms exist, in pharmaceutically acceptable salt, ester, and prodrug form. In addition, the therapeutically effective amount can be administered in a dosage unit form that is constant, or can vary with individual patient needs. Preferably, the therapeutically effective amount of the pyrophosphate-type compound is administered in a pharmaceutically acceptable carrier or medium. Additional excipients may be administered with the pyrophosphate-type compound.

In one embodiment, the pyrophosphate-type compound is administered to the individual in a dialysate fluid, or during dialysis. The pyrophosphate-type compound can be administered to the individual in a dialysate fluid at a concentration of pyrophosphate-type compound of at least about 1 μM. The pyrophosphate concentration can be from about 1 μM to about 10 μM, or from about 3 μM to about 5 μM.

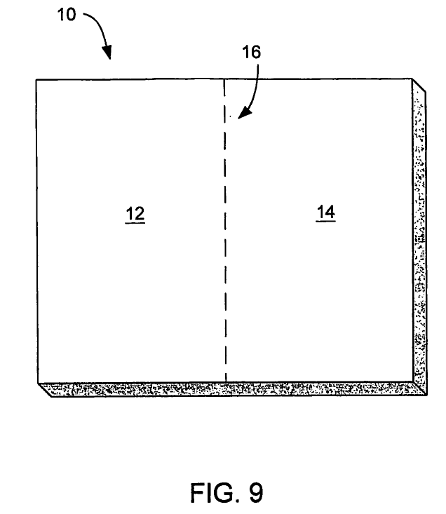

Included in the present disclosure are hemodialysis systems. One exemplary hemodialysis system is depicted in FIG. 9. The hemodialysis system 10 shown in FIG. 9 includes a blood compartment 12, a dialysate compartment 14, the blood compartment 12 and the dialysate compartment 14 being separated by a membrane 16. The membrane 16 creates a semipermeable fluidic communication path between the blood compartment 12 and the dialysate compartment 14. The dialysates described herein are disposed within the dialysate compartment 14 and from there can diffuse into the blood compartment 12, which is re-circulated to an individual, thereby administering a pyrophosphate-type compound to the individual. It should be noted that the hemodialysis system 10 depicted in FIG. 9 is an extremely simplified block diagram version that is merely intended to illustrate the principles of the disclosed compositions and methods.

Pyrophosphate Levels in Hemodialysis Patients

Pyrophosphate (PPi) is a known inhibitor of hydroxyapatite formation and has been shown to inhibit medial vascular calcification in vitamin D-toxic rats. It has been demonstrated that endogenous production of PPi prevents calcification of rat aorta cultured in high concentrations of Ca and PO₄. To determine whether PPi metabolism is altered in hemodialysis patients, plasma levels and dialytic clearance of PPi were measured in stable hemodialysis patients. Predialysis plasma samples were obtained from 15 patients in an outpatient dialysis unit and from 23 inpatients. The inpatients were clinically stable and were admitted for transplant evaluation or for dialysis access problems. Plasma [PPi] was 2.26+/−0.19 μM compared to 3.26+/−0.17 in 36 normal subjects (p<0.01). Approximately 3 0% was protein bound and this was not altered in dialysis patients. There was only a weak inverse correlation with age, and levels did not vary between interdialytic periods of 2 and 3 days. Plasma [PPi] decreased 32+/−5% after standard hemodialysis in 17 patients. In vitro clearance of PPi by a 2.1 m² cellulose acetate dialyzer was 36% and the mean PPi removal in 5 patients was 43±5 μmoles, consistent with a similar in vivo clearance. Cleared PPi was greater than the plasma pool but less than the estimated extracellular fluid pool. Erythrocyte PPi content decreased 24+/−4%, indicating that intracellular PPi is removed as well. As a result, it was concluded that plasma [PPi] is reduced in hemodialysis patients and that PPi is cleared by dialysis. Plasma levels in some patients were below those we have previously shown to prevent calcification of vessels in culture, suggesting that altered PPi metabolism could contribute to vascular calcification in hemodialysis patients.

Rat aortas fail to calcify when cultured in very high calcium and phosphate concentrations, and that this is due to an inhibitory effect of pyrophosphate produced by the vessels. This inhibition occurred at PPi concentrations normally present in human plasma. PPi is well established as an inhibitor of calcification in cartilage and of calcium oxalate crystallization in the kidney, and inhibits vascular calcification in vitamin D-toxic rats. It is a direct and potent inhibitor of hydroxyapatite formation in vitro and even the small concentrations in plasma (2-4 M) are sufficient to completely prevent crystallization from saturated solutions of calcium and phosphate. Humans with low levels of PPi due to the absence of a PPi-producing enzyme develop severe, fatal arterial calcification that can be prevented by therapy with bisphosphonates (also known as diphosphonates), which are nonhydrolyzable analogs of PPi. These findings suggest that vascular calcification cannot occur in the presence of normal concentrations of pyrophosphate and that the medial vascular calcification in ESRD must be associated with altered pyrophosphate metabolism.

Comparison of plasma [PPi] in normal subjects and in hemodialysis patients (predialysis) is shown in Table 1 below. The mean concentration was 31% lower in hemodialysis patients. Because the dialysis patients were significantly older due to an elderly subpopulation not represented in the normals, data were also analyzed for age less than 60. Plasma [PPi] was still lower in hemodialysis patients despite similar ages (47 vs. 41 in normals, p=NS).

TABLE 1
Plasma pyrophosphate levels in normal subjects and
in hemodialysis patients nor to dialysis.

| | normal subjects | | | hemodialysis patients | | | |
|---|---|---|---|---|---|---|---|
| | | | | | | | p vs. |
| | n | mean | Std. error | n | mean | Std. error | normals |
| all | 36 | 3.26 | 0.17 | 38 | 2.26 | 0.19 | <0.001 |
| age < 60 | 34 | 3.24 | 0.18 | 19 | 1.98 | 0.27 | <0.001 |

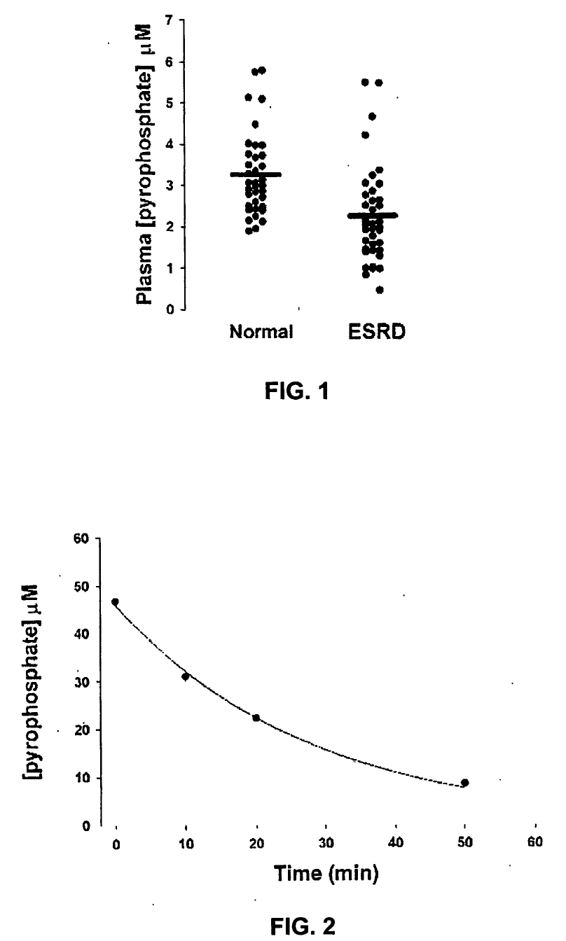

As shown in FIG. 1, the reduced mean plasma [PPi] was due to a subset of patients with very low levels. Whereas the highest levels in the normal individuals and the hemodialysis patients were similar, 15 patients had levels below the lowest level in the normal subjects. The effect of other parameters on plasma [PPi] in hemodialysis patients is shown in Table 2 below.

TABLE 2
Plasma pyrophosphate levels in hemodialysis patients prior to dialysis.

| | n | mean | Std. error | median |
|---|---|---|---|---|
| all | 38 | 2.26 | 0.19 | 2.01 |
| inpatient | 23 | 2.45 | 0.26 | 2.13 |
| outpatient | 15 | 1.95 | 0.27 | 1.92 |
| 2 day interdialytic | 25 | 2.24 | 0.25 | 1.91 |
| 3 day interdialytic | 12 | 2.22 | 0.31 | 2.09 |

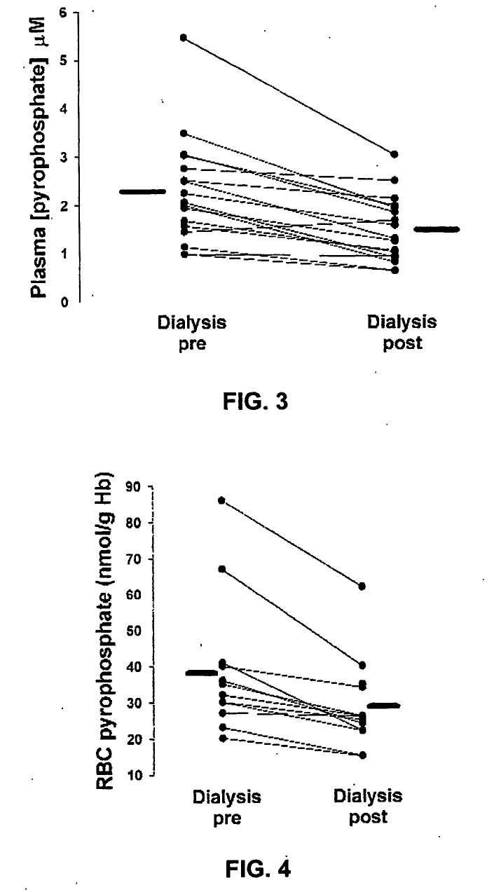

Several studies were undertaken to determine the extent of PPi removal with dialysis. In vitro PPi clearance was determined by dialyzing a 4 L solution of PPi in physiologic saline at a flow of 400 ml/min against a standard clinical dialysate without calcium (to prevent precipitation of PPi) at a flow of 800 ml/min using a 2.1 m² cellulose acetate membrane. As shown in FIG. 2, the disappearance of PPi fit a single exponential function and revealed a dialyzer clearance of 36%. In 17 patients, some of whom were included in the predialysis data, plasma PPi concentration was measured before and after dialysis (FIG. 3). The level decreased in all but one patient with a mean decrease of 32±2.7%, but the range was large (4% to 59%, excluding the one patient in whom there was an increase). Dialysis decreased erythrocyte PPi content in 12 of 13 patients, with the level unchanged in the other patient (FIG. 4). The mean decrease was 24+7%.

Dialysate was collected during 4 treatments in 4 different patients order to measure the total amount of PPi removed. The total amounts cleared in these treatments were (in μmoles) 42, 42, 32, and 57. The mean value was 43+5 μmoles. Despite the fact that the kidney normally clears PPi, plasma levels were reduced in hemodialysis patients. Further compounding the reduced plasma [PPi] is its clearance by dialysis, resulting in a further 32% decrease. Thus, at the end of dialysis, levels were approximately half the normal level.

The reduced pyrophosphate levels in hemodialysis patients and the further decrease during dialysis have important implications since PPi is a potent inhibitor of hydroxyapatite crystallization. The concentration in normal plasma [PPi] prevents crystallization from supersaturated solutions of calcium and phosphate. We have previously shown that this concentration also prevents calcification of rat aortas in culture. See Lomashvili K A, Cobbs S, Hennigar R A, Hardcastle K J, O'Neill W C: Phosphate-induced vascular calcification: role of pyrophosphate and osteopontin. J Am Soc Nephrol 15:1392-1401, 2004. Thus, the reduced levels in hemodialysis patients can promote hydroxyapatite formation to occur. Administration of PPi to vitamin D-toxic rats inhibits vascular calcification (see Schibler D, Russell G G, Fleisch H: Inhibition by pyrophosphate and polyphosphate of aortic calcification induced by vitamin D₃ in rats. Clin Sci 35:363-372, 1968), suggesting that PPi or bisphosphonate analogs can be therapeutic.

Pyrophosphate as Inhibitor of Vascular Calcification

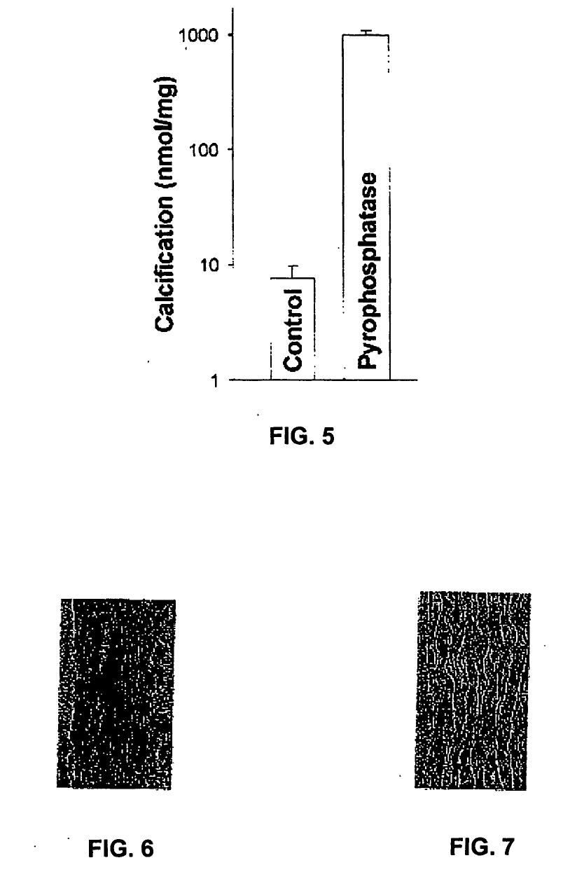

Pyrophosphate was also investigated as a possible inhibitor of calcification by studying rat aortic rings. It is not present in DMEM medium (Mediatech, Herndon, Va., USA), but its concentration after three days of culturing aortic rings was 0.44+/−0.03 μM (one aortic ring in 500 μL of medium), indicating that it was produced by aortas. These measurements were made in normal DMEM to avoid sequestration of pyrophosphate in calcium phosphate deposits. Elimination of pyrophosphate by adding inorganic pyrophosphatase (as judged from the disappearance of [³²P]pyrophosphate, not shown) induced calcification of normal aortas (FIG. 5). Focal medial calcification was apparent with hematoxylin and eosin staining (FIG. 6), and von Kossa staining revealed calcification of some elastin fibers (FIG. 7).

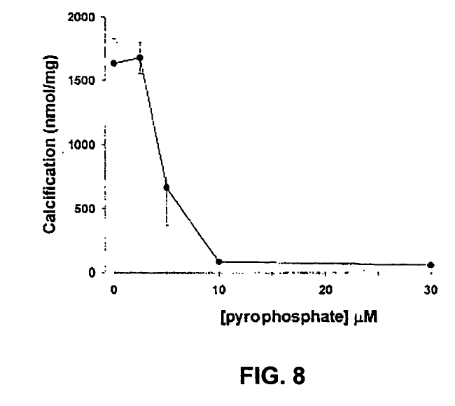

Addition of pyrophosphate prevented calcification in injured aortas (FIG. 8), confirming that pyrophosphate inhibits medial calcification. There was no inhibition with 2.5 M but almost complete inhibition with 10 μM pyrophosphate. Based on the rate of hydrolysis of [³²P]pyrophosphate in aortic cultures (not shown), the estimated concentrations 3 days after adding 5, 10, and 30 μM pyrophosphate were 18 μM, 3.1 μM, and 7.9 μM, respectively. Thus, the inhibition of calcification by pyrophosphate is actually more potent than indicated in the FIG. 8. The appearance rate of pyrophosphate in culture medium was substantially reduced in injured aortas (36+4 μmol/mg/d, n=12, vs. 145+8 μmol/mg/d in normal aortas, n−22) and alkaline phosphatase activity was significantly increased in injured aortas (1.16+0.17 units/mg, n=15 vs. 0.43+0.04 units/mg, n=12, in uninjured aorta).

This study demonstrates that medial calcification can be induced in intact rat aorta cultured with alkaline phosphatase or inorganic pyrophosphatase. The calcification is in the form of hydroxyapatite, requires a high PO4⁻³ concentration, and is histologically similar to the calcification observed in vessels from uremic patients and rats with chronic renal failure. Rat aortas cultured without these enzymes and not subjected to injury exhibited no calcification in the high-PO4⁻³ medium, even up to 21 days in culture. The small, initial incorporation of ⁴⁵Ca under normal conditions presumably represents equilibration with intracellular Ca and Ca normally bound to extracellular matrix since it did not increase over time. Concentrations of both Ca²⁺ and PO4⁻³ are elevated in high-PO4⁻³ medium compared to human serum and, based on free concentrations, would be equivalent to a total calcium-phosphorus product in human serum of 180 mg²/dl², which is well above generally accepted clinical thresholds. Thus an elevated calcium-phosphorus product is not sufficient to produce medial calcification in vitro. Vascular calcification is a chronic process in vivo and we cannot rule out the possibility that longer culture times are required to observe calcification of normal vessels in vitro. However, the absence of any increase in ⁴⁵Ca deposition over 3 weeks argues against this.

The absence of calcification was due to inhibitory activity in normal aortas and this inhibition, can be explained by the release of pyrophosphate from smooth muscle. Alkaline phosphatase and inorganic pyrophosphatase induced calcification of normal aortas and pyrophosphate inhibited calcification of injured aortas. Pyrophosphate inhibits hydroxyapatite formation in vitro and exogenous pyrophosphate inhibits aortic calcification in rats given large doses of vitamin D₃. Bisphosphonates, which are analogs of pyrophosphate, exhibit the same properties. It is likely that the inhibition by endogenous pyrophosphate demonstrated in cultured rat aortas also occurs in vivo since the concentration that maximally inhibited calcification in injured aortas (approximately 3 μM) is similar to that reported for normal human plasma. Furthermore, deficiency of PC-1, an ecto-ATPase that produces pyrophosphate, results in reduced plasma pyrophosphate levels and extensive arterial calcification in humans, which can be prevented with bisphosphonate therapy. Mice lacking ANK, a putative pyrophosphate transporter, exhibit reduced pyrophosphate production and extensive ectopic calcification, although not in vessels.

Addition of Pyrophosphate to Dialysate in Hemodialysis Treatment

Pyrophosphate, a small, dialyzable molecule present in normal blood, is a potent inhibitor of vascular calcification in vitro. There is also strong but indirect evidence that pyrophosphate inhibits vascular calcification in vivo, including in humans. Our in vitro studies indicate that this inhibition occurs at concentrations normally present in human plasma (3-5 μM). Our recent studies have shown that plasma pyrophosphate levels are reduced in hemodialysis patients and are reduced even further during hemodialysis. Addition of pyrophosphate to dialysate should prevent the net loss of pyrophosphate in the blood of patients undergoing dialysis, and could reduce or prevent vascular calcification in hemodialysis patients.

Accordingly, the disclosure includes compositions of dialysate concentrate having pyrophosphate at a concentration of greater than about 50 μM and less than about 1 mM. In a standard 45× dialysis system, the bicarbonate concentrate is diluted about 25× with water and acid concentrate to yield the final dialysate. Also included are final compositions of dialysate comprising pyrophosphate concentrations of at least about 1 μM. The dialysate concentration of pyrophosphates can be from about 1 μM to about 10 μM, or from about 3 μM to about 5 μM, wherein the final composition is the dialysis composition to which the hemodialysis patient is exposed.

Also included are methods for reducing or preventing vascular calcification having administering dialysate to patients wherein the final dialysate comprises a pyrophosphate concentration of at least about 1 μM. The pyrophosphate concentration can be from about 1 μM to about 10 μM, or from about 3 μM to about 5 μM, wherein the final composition is the dialysis composition to which the hemodialysis patient is exposed.

Different dialysis systems work in different ways. The present disclosure is intended to cover methods and compositions wherein the final dialysate includes a pyrophosphate concentration of at least about 1 μM. The pyrophosphate concentration can be from about 1 μM to about 10 μM, or from about 3 μM to about 5 μM. The final pyrophosphate concentration can be reached in different dialysis systems in a number of different ways, for example: (1) via dilution of a basic concentrate containing pyrophosphate. Typically, basic dialysate concentrates are diluted about 25-fold, although the range is typically 20- to 30-fold. Accordingly, the concentration of pyrophosphate in the basic concentrate would typically range from about 60 μM to about 150 μM; (2) via dilution of a powdered concentrate containing pyrophosphate. Either the acidic bath or basic bath, or both, can be obtained via solubilization and dilution of a solid (e.g., powder, granular, and crystalline) composition containing pyrophosphate; and (3) via dilution of an acidic bath concentrate containing pyrophosphate. Typically, acid bath concentrates are diluted by a factor between 30-fold and 45-fold. Accordingly, the concentration of pyrophosphate in the acid concentrate would typically range from about 90 μM to about 225 μM.

Also covered by the disclosure are methods for reducing or preventing vascular calcification that include administering dialysate to patients wherein the dialysate includes a pyrophosphate concentration of at least about 1 μM. The pyrophosphate concentration can be from about 1 μM to about 10 μM, or from about 3 μM to about 5 μM, and a bicarbonate concentration from about 10 mM to about 100 mM, wherein the final composition is the dialysis composition to which the hemodialysis patient is exposed.

The disclosure contemplates incorporation of sodium pyrophosphate into dialysate. Sodium pyrophosphate can be combined with other pyrophosphate salts as well. For example, sodium pyrophosphate can be combined with ferric pyrophosphate, which may have the added benefit of providing the body with soluble iron. This disclosed compositions and methods provides a significant advantage over the prior art by preventing depletion of pyrophosphate in hemodialysis patients, and thereby preventing, reducing, or potentially reversing vascular calcification.

### EXAMPLE 1

A pyrophosphate-bicarbonate dialysate concentrate was prepared, including sodium pyrophosphate (125 μM) and sodium bicarbonate (967 mM). Dialysate is normally constituted during hemodialysis by the mixing of two concentrated solutions (acid bath concentrate and basic bath concentrate) with appropriate amounts of water. Pyrophosphate was added to the bicarbonate concentrate. Pyrophosphate was found to be stable and soluble at a concentration of 125 μM in the bicarbonate solution. The pyrophosphate remained soluble after the bicarbonate concentrate was diluted and combined with the acid dialysate solution to yield the final dialysate solution.

### EXAMPLE 2

A pyrophosphate-bicarbonate dialysate concentrate was prepared, including sodium pyrophosphate (125 μM) and sodium bicarbonate (967 mM). Pyrophosphate was found to be stable and soluble at a concentration of 125 μM in the bicarbonate solution. The pyrophosphate remained soluble after the bicarbonate concentrate was diluted and combined with an acidic dialysate to yield the final dialysate.

The resulting final dialysate is used to perform hemodialysis in a human with kidney disease. The patient experiences reduced calcium deposition relative to what would have been experienced had the patient been treated with conventional hemodialysis solutions lacking pyrophosphate.

### EXAMPLE 3

A pyrophosphate-bicarbonate dialysate concentrate is prepared, including sodium pyrophosphate (100 μM) and sodium bicarbonate (967 mM). The basic dialysate is diluted with water, then mixed with the acid dialysate to yield the final dialysate. The resulting final dialysate is used to perform hemodialysis in a human with kidney disease.

### EXAMPLE 4

A pyrophosphate-bicarbonate dialysate concentrate is prepared, including sodium pyrophosphate (75 μM) and sodium bicarbonate (967 mM). The basic dialysate is diluted 25-fold with water, then mixed with the acid dialysate to yield the final dialysate. The resulting final dialysate is used to perform hemodialysis in a human with kidney disease.

### EXAMPLE 5

A pyrophosphate-bicarbonate dialysate concentrate is prepared, including sodium pyrophosphate (90 μM), ferric pyrophosphate (10 μM) and sodium bicarbonate (967 mM). The basic dialysate is diluted 25-fold with water, then mixed with the acid dialysate to yield the final dialysate. The resulting final dialysate is used to perform hemodialysis in a human with kidney disease.

### EXAMPLE 6

An acidic dialysate concentrate is prepared using standard ingredients in addition to sodium pyrophosphate (136 μM). The acid dialysate concentrate is diluted 34-fold with water, then mixed with the basic dialysate to yield the final dialysate. The resulting final dialysate is used to perform hemodialysis in a human with kidney disease.

It should be emphasized that the above-described embodiments of the present disclosure are merely possible examples of implementations, and are set forth only for a clear understanding of the principles of the disclosure. Many variations and modifications may be made to the above-described embodiments of the disclosure without departing substantially from the spirit and principles of the disclosure. All such modifications and variations are intended to be included herein within the scope of this disclosure and protected by the following claims.

The following is claimed:

1. A method of providing vascular calcification therapy to an individual in need of treatment comprising the step of administering to the individual an effective amount of a pyrophosphate-type compound.

2. The method of claim 1, wherein the pyrophosphate-type compound is an alkali metal pyrophosphate.

3. The method of claim 1, wherein the pyrophosphate-type compound is chosen from tetraalkali metal pyrophosphate, dialkali metal diacid pyrophosphate, trialkali metal monoacid pyrophosphate, and mixtures thereof.

4. The method of claim 1, wherein the pyrophosphate-type compound is chosen from tetrasodium pyrophosphate, tetrapotassium pyrophosphate, dicalcium pyrophosphate, phosphoric acid, sodium acid pyrophosphate, sodium dihydrogen pyrophosphate, and mixtures thereof.

5. The method of claim 1, wherein the vascular calcification is caused by renal disease or failure.

6. The method of claim 1, further comprising treating the individual with dialysate.

7. The method of claim 1, wherein the pyrophosphate-type compound is administered to the individual in a dialysate fluid.

8. The method of claim 1, wherein the pyrophosphate-type compound is administered to the individual during dialysis.

9. The method of claim 1, wherein the pyrophosphate-type compound has the following structural formula: 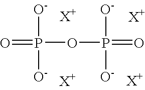
wherein the X is chosen from at least one of a hydrogen and a cation.

10. The method of claim 9, wherein each X is individually chosen to be at least one of: hydrogen, sodium, potassium, and calcium.

11. The method of claim 1, wherein the pyrophosphate-type compound is administered to the individual in a dialysate fluid at a concentration of pyrophosphate-type compound of at least about 1 μM.

12. The method of claim 1, wherein the pyrophosphate-type compound is administered to the individual in a dialysate fluid at a concentration of pyrophosphate-type compound from about 1 μM to about 10 μM.

13. The method of claim 1, wherein the pyrophosphate-type compound is administered to the individual in a dialysate at a concentration from about 3 μM to about 5 μM.

14. A method of prophylactically treating vascular calcification comprising administering to an individual in need of treatment an effective amount of at least one pyrophosphate-type compound.

15. The method of claim 14, wherein the pyrophosphate-type compound is an alkali metal pyrophosphate.

16. The method of claim 14, wherein pyrophosphate-type compound is administered to the individual in a dialysate.

17. A pharmaceutical composition comprising at least one pyrophosphate-type compound in combination with a pharmaceutically acceptable carrier, wherein the at least one pyrophosphate-type compound is present in a dosage level effective to treat vascular calcification.

18. The pharmaceutical composition of claim 17, wherein the at least one pyrophosphate-type compound is an alkali metal pyrophosphate.

19. The pharmaceutical composition of claim 17, wherein the at least one pyrophosphate-type compound includes pharmaceutically acceptable salts of the pyrophosphate-type compound.

20. The pharmaceutical composition of claim 17, wherein the at least one pyrophosphate-type compound includes pharmaceutically acceptable prodrugs of the pyrophosphate-type compound.

21. The pharmaceutical composition of claim 17, wherein the pharmaceutically acceptable carrier is a dialysate.

22. A method of hemodialyzing an individual in need thereof, comprising the steps of:
diffusing dialysate comprising at least one pyrophosphate-type compound across a membrane in a hemodialysis system; and
exposing the individual to an effective amount of the pyrophosphate-type compound.

23. The method of claim 22, wherein the pyrophosphate-type compound is an alkali metal pyrophosphate.

24. The method of claim 22, further comprising treating vascular calcification in the individual through exposing the individual to an effective amount of the pyrophosphate-type compound.

25. A dialysate concentrate comprising at least one pyrophosphate-type compound.

26. The dialysate concentrate of claim 25, wherein the at least one pyrophosphate-type compound has the formula of the following structure: 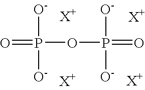
wherein the X is chosen from at least one of a hydrogen and a cation.

27. The dialysate concentrate of claim 26, wherein each X is individually chosen to be at least one of: hydrogen, sodium, potassium, and calcium.

28. The dialysate concentrate of claim 25, wherein the pyrophosphate-type compound is present in the dialysate concentrate at a concentration of about 50 μM to about 1 mM.

29. The dialysate concentrate of claim 25, wherein the at least one pyrophosphate-type compound has the formula of the following structure: 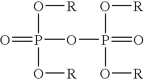
wherein R is individually chosen to be a functional group of at least one of hydrogen, alkyl groups, aryl groups, halo groups (F, Cl, Br, and I) hydroxy groups, alkoxy groups, alkylamino groups, dialkylamino groups, acyl groups, carboxyl groups, carboamido groups, sulfonamide groups, aminoacyl groups, amide groups, amine groups, nitro groups, organo selenium compounds, hydrocarbons, cyclic hydrocarbons, hydrogen, nitrogen, oxygen, sulphur, NR, and CR.

30. A hemodialysis system, comprising:
a blood compartment;
a membrane in fluidic communication with the blood compartment; and
a dialysate compartment, the dialysate compartment comprising a dialysate comprising a pyrophosphate-type compound.

31. The hemodialysis system of claim 30, wherein the pyrophosphate-type compound is an alkali metal pyrophosphate.

32. The hemodialysis system of claim 30, wherein the pyrophosphate-type compound has the formula of the following structure: 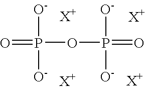
wherein the X is chosen from at least one of a hydrogen and a cation.

33. The hemodialysis system of claim 32, wherein each X is individually chosen to be at least one of: hydrogen, sodium, potassium, and calcium.

34. The hemodialysis system of claim 30, wherein the pyrophosphate-type compound is administered to the individual in a dialysate at a concentration of at least about 1 μM.
